(19) United States
(12) Patent Application Publication
(10) Pub. No.: US 2012/0258093 A1
(43) Pub. Date: Oct. 11, 2012

(54) VLA-4 AS A BIOMARKER FOR PROGNOSIS AND TARGET FOR THERAPY IN DUCHENNE MUSCULAR DYSTROPHY

(75) Inventors: Gillian Butler-Browne, Paris (FR); Suse Dayse Silva-Barbosa, Rio de Janeiro (BR); Wilson Savino, Rio de Janeiro (BR); Alexandra Prufer De Queiroz Campos Araujo, Rio de Janeiro (BR); Fernanda Pinto-Mariz, Rio de Janeiro (BR); Luciana Rodrigues Carvalho, Rio de Janeiro (BR); Thomas Voit, Paris (FR)

(73) Assignee: Institut National De La Sante Et De La Recherche Medicale (INSERM), Paris (FR)

(21) Appl. No.: 13/390,236

(22) PCT Filed: Aug. 19, 2010
(86) PCT No.: PCT/EP2010/062092

§ 371 Date: Jun. 28, 2012

(30) Foreign Application Priority Data
Aug. 20, 2009 (EP) 09305772.7

(51) Int. Cl. G01N 21/64; C12Q 1/68; A61K 31/713; G01N 33/566; A61K 39/395; A61K 31/7088; C40B 30/04; A61K 38/02

(52) U.S. Cl. 424/133.1; 506/9; 435/6.12; 435/6.11; 514/1.1; 435/7.24; 424/130.1; 514/44 R; 514/44 A

(57) ABSTRACT

The invention relates to methods for the treatment of Duchenne muscular dystrophy and to methods for determining the prognosis of a subject affected with Duchenne Muscular Dystrophy. More particularly, the present invention relates to a VLA-4 antagonist for use in the treatment of Duchenne Muscular Dystrophy. The present invention also relates to a method for determining the prognosis of a subject affected with Duchenne Muscular Dystrophy wherein said method comprising a step consisting of determining the level of VLA-4 high T cells in a blood sample obtained from said subject.

### FIELD OF THE INVENTION

The invention relates to methods for the treatment of Duchenne muscular dystrophy and methods for determining the prognosis of a subject affected with Duchenne Muscular Dystrophy.

### BACKGROUND OF THE INVENTION

The muscular dystrophies are a group of clinically and genetically heterogeneous myopathies characterized by progressive degenerative changes in the skeletal muscles. This group of genetically distinct disorders shares clinical and pathological characteristics but varies in severity, inheritance pattern, and molecular defects.

Duchenne muscular dystrophy (DMD) is the most common of these disorders, affecting 1 in 3,500 male births. DMD is caused by mutations or deletions in the dystrophin gene (chromosome Xp21) leading to its reduction at the mRNA level and absence at the protein level. This loss of dystrophin causes a fragility of the muscle membrane resulting in repeated rounds of muscle fiber necrosis and regeneration as well as progressive replacement of the muscle fibers by fibrosis and fat in the later stages of the disease. Subjects with DMD present a progressive muscle weakness resulting in a loss of ambulation usually in the early teens. Respiratory failure and cardiomyopathy are also present and death occurs, generally during the third decade of life.

However, studies in animal models and in DMD subjects seem to suggest that the immune system could also contribute to the lesions observed in the skeletal muscles. An increased inflammation has been described in dystrophin-deficient muscles, and it has been shown that the in vivo depletion of CD8⁺ T cells in the mdx mouse (the murine natural model of DMD) or the impairment of T cell cytotoxicity by the removal of perforin attenuates the disease. It has also been shown that irradiation of prenecrotic mdx mice improves or delays the pathological symptoms, presumably due to a decrease in the number of immune cells that can invade and kill the muscle. Finally, adoptive transfer of mdx immune cells in combination with muscle extracts resulted in muscle pathology in health murine recipients.

Evidence in humans has also suggested that the immune system plays an important role in the disease pathophysiology. Clonal populations of lymphocytes with conserved T cell receptor sequences have been identified in DMD biopsies, suggesting that they have been activated and expanded polyclonally. In addition, the treatment with glucocorticoids can improve the overall motor function and is associated with a reduction in the number of inflammatory mononuclear cells, mainly CD8 T cells, and dendritic cells, with a positive correlation between the reduction in the number of dendritic cells and clinical improvement.

Taken together these data strongly suggest that T cells are involved in the pathophysiology of DMD. However, the mechanisms that may contribute and regulate the migration and perpetuation of this immune response in the muscle tissue remain to be clarified.

Interactions between the extracellular matrix (ECM) ligands and receptors have been shown to be important for cell migration in different physiological and pathological conditions. An enhancement in the expression types I and IV collagens and laminin has been observed in the skeletal muscles of mdx mice. These alterations were accompanied by an important inflammatory infiltrate in the adjacent area. An increased expression of ECM receptors (VLA-4, VLA-5 and VLA-6) on the surface of inflammatory cells close to the regions of necrosis was also demonstrated (Lagrota-Cândido et al, 1999). In the skeletal muscles of subjects with DMD it is well established that there is an increase in the ECM. Taken together, these data suggest that modifications in the expression of ECM receptors and ligands may contribute both to the migration of cells and to the maintenance of the local inflammation.

Therefore, it is relevant to identify the molecules involved in the migration and retention of the immune cells within the muscle tissue. By improving knowledge of the molecular mechanisms responsible for the clinical symptoms of DMD this may help to identify novel therapeutic targets and develop new approaches that could improve the quality of life of these subjects.

### SUMMARY OF THE INVENTION

The present invention relates to a VLA-4 antagonist for use in the treatment of Duchenne Muscular Dystrophy.

The present invention also relates to an inhibitor of expression of a gene encoding a VLA-4 subunit for use in the treatment of Duchenne Muscular Dystrophy.

The present invention also relates to a pharmaceutical composition comprising a VLA-4 antagonist or an inhibitor of expression according to the invention for use in the treatment of Duchenne Muscular Dystrophy.

The present invention also relates to a method for determining the prognosis of a subject affected with Duchenne Muscular Dystrophy wherein said method comprising a step consisting of determining the level of VLA-4^{high} T lymphocytes in a blood sample obtained from said subject.

The present invention also relates to a method for determining the prognosis of a subject affected with Duchenne Muscular Dystrophy wherein said method comprises the step of analyzing a biological sample from said subject for:
i) detecting the presence of a mutation in the gene encoding CD49d (alpha4 integrin chain) and/or CD29 (beta1 integrin chain) of VLA-4, and/or
ii) analyzing the expression of the gene encoding CD49d and/or CD29 of VLA-4.

### DETAILED DESCRIPTION OF THE INVENTION

In the present study the inventors have followed a cohort of subjects with DMD at different stages of their disease. They demonstrate that the level of expression of VLA-4 integrin both on CD4⁺ and on CD8⁺ T lymphocytes can be correlated with the severity or progression of the disease and that an increased membrane level of VLA4 integrin expression is also involved in the increased ex-vivo migratory responses of the T lymphocytes. Furthermore they present evidence that an increased membrane level of VLA4 is associated with an increase of VLA4 expressing cells in the muscle specimens of DMD patients, suggesting that increased transmigration into the diseased muscle is also a phenomenon that occurs in vivo. Most importantly, they have shown that this increased migration can be inhibited ex-vivo using an anti-CD49d antibody. The results show that VLA4 is not only a good prognostic marker for DMD, but could also provide a new therapeutic target to slow down degeneration fatty infiltration and fibrosis in DMD, and thereby stabilise muscle function.

### Therapeutic Methods and Use

The present invention provides methods and compositions (such as pharmaceutical compositions) for treating or preventing Duchenne Muscular Dystrophy.

According to a first aspect, the invention relates to a VLA-4 antagonist for use in the treatment of Duchenne Muscular Dystrophy.

As used herein, the term “VLA-4” has its general meaning in the art and refers to Integrin alpha4beta1 (Very Late Antigen-4), also known as CD49d/CD29. This integrin is an alpha/beta heterodimeric glycoprotein in which the alpha-4 subunit, named CD49d, is noncovalently associated with the beta-1 subunit, named CD29. The cell membrane molecule VCAM-1 (vascular cell adhesion molecule 1) and fibronectin (which is an extracellular matrix protein) bind to the integrin VLA-4, which can be normally expressed on leukocyte plasma membranes. The term may include naturally occurring VLA-4s and variants and modified forms thereof. The VLA-4 can be from any source, but typically is a mammalian (e.g., human and non-human primates) VLA-4, particularly a human VLA-4.

The term “VLA-4 antagonist” has its general meaning in the art and includes any chemical or biological entity that, upon administration to a subject, results in inhibition or down-regulation of a biological activity associated with activation of the VLA-4 in the subject, including any of the downstream biological effects otherwise resulting from the binding to VLA-4 to its natural ligands (e.g. VCAM-1 or fibronectin). In general, VLA-4 antagonists are well known in the art, and comprise any agent that can block VLA-4 activation or any of the downstream biological effects of VLA-4 activation. For example, such a VLA-4 antagonist can act by occupying the binding site or a portion thereof of the VLA-4, thereby making the receptor inaccessible to its natural ligand (e.g. VCAM-1 or fibronectin) so that its normal biological activity is prevented or reduced. In the context of the present invention, VLA-4 antagonists are preferably selective for the VLA-4 as compared with the other VLA (VLA-1, VLA-2, VLA-3 and VLA-5). By “selective” it is meant that the affinity of the antagonist for the VLA-4 is at least 10-fold, preferably 25-fold, more preferably 100-fold, still preferably 500-fold higher than the affinity for other VLAs. The antagonistic activity of compounds towards the VLA-4 may be determined using various methods well known in the art. For example, the agents may be tested for their capacity to block the interaction of VLA-4 receptor cells bearing a natural ligand of VLA-4 (e.g. VCAM-1 or fibronectin), or purified natural ligand of VLA-4 (e.g. VCAM or fibronectin). Typically, the assay can be performed with VLA-4 and VCAM-1 expressed on the surface of cells, or with the VLA-4 mediated interaction with extracellular fibronectin or purified or recombinant VCAM-1.

In its broadest meaning, the term “treating” or “treatment” refers to reversing, alleviating, inhibiting the progress of, or preventing the disorder or condition to which such term applies, or one or more symptoms of such disorder or condition.

In one embodiment, the VLA-4 antagonist may be a low molecular weight antagonist, e.g. a small organic molecule.

The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

Exemplary small organic molecules that are VLA-4 antagonists include but are not limited to those described in U.S. Pat. Nos. 6,407,06; 5,998,447; 6,034,238; 6,306,887; 6,355,662; 6,432,923; 6,514,952; 6,514,952; 6,667,331; 6,668,527; 6,794,506; 6,838,439; 6,838,439; 6,903,128; 6,953,802; 7,205,310; 7,223,762; 7,320,960; 7,514,409; 7,538,215 and in US Patent Application Publications Numbers US 2002/0049236; US 2002/0052470; US 2003/0087956; US 2003/0144328; US 2004/0110945; US 2004/0220148; US 2004/0266763; US 2005/0085459 US 2005/0222119; US 2007/0099921; US 2007/0129390; US 2008/0064720; US 2009/0048308; US 2009/0069376; US 2009/0192181 and US 2010/0016345 that are hereby incorporated by reference into the present disclosure.

Another example of VLA-4 antagonist includes R411 (N-(2-Chloro-6-methylbenzoyl)-4-[(2,6-dichlorobenzoyl)amino]-L-phenylalanine-2-(diethylamino)ethyl ester) that is an ester pro-drug of the active moiety, N-(2-chloro-6-methylbenzoyl)-4-[(2,6-dichlorobenzoyl)amino]-L-phenylalanine R411 has the following chemical structure: R411 is disclosed in U.S. Pat. No. 6,229,011, which disclosure is incorporated by reference herein.

Another example of VLA-4 antagonist includes trans-4-[1-[[2,5-dichloro-4-(1-methyl-3-indolylcarboxamido)phenyl]acetyl]-(4S)-methoxy-(2S)-pyrrolidinylmethoxy]cyclohexanecarboxylic acid as described in Muro F et al. Muro F, Iimura S, Sugimoto Y, Yoneda Y, Chiba J, Watanabe T, Setoguchi M, Iigou Y, Matsumoto K, Satoh A, Takayama G, Taira T, Yokoyama M, Takashi T, Nakayama A, Machinaga N. Discovery of trans-4-[1-[[2,5-Dichloro-4-(1-methyl-3-indolylcarboxamido)phenyl]acetyl]-(4S)-methoxy-(2S)-pyrrolidinylmethoxy]cyclohexanecarboxylic acid: an orally active, selective very late antigen-4 antagonist. J Med. Chem. 2009 Dec. 24; 52(24):7974-92).

Another example of VLA-4 antagonist includes N-{N-[(3-cyanobenzene)sulfonyl]-4(R)-(3,3-difluoropiperidin-1-yl)-(1)-prolyl}-4-[(3′,5′-dichloro-isonicotinoyl)amino]-(1)-phenylalanine (MK-0617) as described in Venkatraman S. et al. (Venkatraman S, Lebsack A D, Alves K, Gardner M F, James J, Lingham R B, Maniar S, Mumford R A, Si Q, Stock N, Treonze K M, Wang B, Zunic J, Munoz B. Discovery of N-{N-[(3-cyanobenzene)sulfonyl]-4(R)-(3,3-difluoropiperidin-1-yl)-(1)-prolyl}-4-[(3′,5′-dichloro-isonicotinoyl)amino]-(1)-phenylalanine (MK-0617), a highly potent and orally active VLA-4 antagonist. Bioorg Med Chem. Lett. 2009 Oct. 1; 19(19):5803-6).

Another example of VLA-4 antagonist includes N-{N-[(3-cyanophenyl)sulfonyl]-4(R)-cyclobutylamino-(L)-prolyl}-4-[(3′,5′-dichloroisonicotinoyl)amino]-(L)-phenylalanine (MK-0668) as described in Lin S. et al. (Lin L S, Lanza T, Jewell J P, Liu P, Jones C, Kieczykowski G R, Treonze K, Si Q, Manior S, Koo G, Tong X, Wang J, Schuelke A, Pivnichny J, Wang R, Raab C, Vincent S, Davies P, Maccoss M, Mumford R A, Hagmann W K. Discovery of N-{N-[(3-cyanophenyl)sulfonyl]-4(R)-cyclobutylamino-(L)-prolyl}-4-[(3′,5′-dichloroisonicotinoyl)amino]-(L)-phenylalanine (MK-0668), an extremely potent and orally active antagonist of very late antigen-4. J Med. Chem. 2009 Jun. 11; 52(11):3449-52.)

Another example of VLA-4 antagonist includes trans-4-[[2-(2-Methylphenylamino)-6-benzoxazolylacetyl]-(4S)-fluoro-(2S)-pyrrolidinylmethoxy]cyclohexanecarboxylic acid as described in Muro F. et al. (Muro F, Iimura S, Yoneda Y, Chiba J, Watanabe T, Setoguchi M, Takayama G, Yokoyama M, Takashi T, Nakayama A, Machinaga N. A novel and potent VLA-4 antagonist based on trans-4-substituted cyclohexanecarboxylic acid. Bioorg Med. Chem. 2009 Feb. 1; 17(3):1232-43).

Another example of VLA-4 antagonist includes 4-[1-[3-chloro-4-[N′-(5-fluoro-2-methylphenyl)ureido]phenylacetyl]-(4S)-fluoro-(2S)-pyrrolidinylmethoxy]benzoic acid as described in Muro F; et al. (Muro F, Iimura S, Yoneda Y, Chiba J, Watanabe T, Setoguchi M, Iigou Y, Takayama G, Yokoyama M, Takashi T, Nakayama A, Machinaga N. Identification of 4-[1-[3-chloro-4-[N′-(5-fluoro-2-methylphenyl)ureido]phenylacetyl]-(4S)-fluoro-(2S)-pyrrolidinylmethoxy]benzoic acid as a potent, orally active VLA-4 antagonist. Bioorg Med. Chem. 2008 Dec. 1; 16(23):9991-10000).

In another embodiment, the VLA-4 antagonist according to the invention is a peptide. For example, the International Patent Application Publication No WO 96/01644 discloses peptides that inhibit binding of VLA-4 to VCAM-1. Other peptides, peptide derivatives or cyclic peptides that bind to VLA-4 and block its binding to VCAM-1 are described in WO 96/22966; WO 96/20216; U.S. Pat. No. 5,510,332; WO 96/00581 or WO 96/06108.

In another embodiment the VLA-4 antagonist may consist in an antibody (the term including antibody fragment) that can block VLA-4 activation.

In particular, the VLA-4 antagonist may consist in an antibody directed against VLA-4 or a ligand of VLA-4 (e.g. VCAM-1 or fibronectin), in such a way that said antibody impairs the binding of said ligand to VLA-4.

Antibodies can be raised according to known methods by administering the appropriate antigen or epitope to a host animal selected, e.g., from pigs, cows, horses, rabbits, goats, sheep, rats and mice, among others. Various adjuvants known in the art can be used to enhance antibody production. Although antibodies useful in practicing the invention can be polyclonal, monoclonal antibodies are preferred. Monoclonal antibodies can be prepared and isolated using any technique that provides for the production of antibody molecules by continuous cell lines in culture. Techniques for production and isolation include but are not limited to the hybridoma technique originally described by Kohler and Milstein (1975); the human B-cell hybridoma technique (Cote et al., 1983); and the EBV-hybridoma technique (Cole et al. 1985). Alternatively, techniques described for the production of single chain antibodies (see, e.g., U.S. Pat. No. 4,946,778) can be adapted to produce anti-VLA-4, or anti-VLA-4 ligands single chain antibodies. VLA-4 antagonists useful in practicing the present invention also include anti-VLA-4, or anti-VLA-4 ligands antibody fragments including but not limited to F(ab′)₂ fragments, which can be generated by pepsin digestion of an intact antibody molecule, and Fab fragments, which can be generated by reducing the disulfide bridges of the F(ab′)₂ fragments. Alternatively, Fab and/or scFv expression libraries can be constructed to allow rapid identification of fragments having the desired specificity to VLA-4.

Humanized antibodies and antibody fragments thereof can also be prepared according to known techniques. “Humanized antibodies” are forms of non-human (e.g., rodent) chimeric antibodies that contain minimal sequence derived from non-human immunoglobulin. For the most part, humanized antibodies are human immunoglobulins (recipient antibody) in which residues from a hypervariable region (CDRs) of the recipient are replaced by residues from a hypervariable region of a non-human species (donor antibody) such as mouse, rat, rabbit or nonhuman primate having the desired specificity, affinity and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. Methods for making humanized antibodies are described, for example, by Winter (U.S. Pat. No. 5,225,539) and Boss (Celltech, U.S. Pat. No. 4,816,397).

Then after raising antibodies directed against VLA-4 as above described, the skilled man in the art can easily select those blocking VLA-4 activation.

Exemplary antibodies that are VLA-4 antagonists include but are not limited to those described in U.S. Pat. No. 6,602,503 and in US Patent Application Publication No US 2003/0185819 that are hereby incorporated by reference into the present disclosure. Also contemplated herein are other antibodies specific for VLA4, including, but not limited to, immunoglobulins described in U.S. Pat. Nos. 6,602,503 and 6,551,593, and published U.S. Application No. 20020197233.

Monoclonal antibodies to the alpha-4 subunit of VLA-4 that block binding to VCAM-1 include HP2/1 (AMAC, Inc. Westbrook Me.), L25 (Clayberger et al., 1987), TY 21.6 (WO 95/19790), TY.12 (WO9105038) and HP2/4. Further antibodies binding to VLA-4 and blocking VCAM-1 binding are described in WO 94/17828. Humanized antibodies to alpha-4 integrin are described by in WO9519790. Another example of humanized monoclonal antibody directed to the alpha-4 subunit of VLA-4 is AN-100226 (Antegren) as described in Elices M J (1998) (Antegren Athena Neurosciences Inc. IDrugs. 1998 June; 1(2):221-7).

Monoclonal antibodies that bind to VCAM-1 and block its interaction with VLA-4 are described in WO 95/30439. Other antibodies to VCAM-1 have been reported by Carlos et al., 1990 and Dore-Duffy et al., 1993.

In a particular embodiment, said VLA-4 antibody is Natalizumab® that is a humanized antibody against VLA-4 as described in U.S. Pat. Nos. 5,840,299 and 6,033,665, which are herein incorporated by reference in their entireties. Natalizumab is a humanized IgG4[kappa] monoclonal antibody directed against the alpha4-integrins alpha4beta1 and alpha4beta7.

In another embodiment the VLA-4 antagonist is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consist of a conformationally constrained antibody variable region displayed by a platform protein, such as E. coli Thioredoxin A that are selected from combinatorial libraries by two hybrid methods (Colas et al., 1996).

Then after raising aptamers directed against the VLA-4 as above described, the skilled man in the art can easily select those blocking VLA-4 activation.

Another aspect of the invention relates to the use of an inhibitor of expression.

An “inhibitor of expression” refers to a natural or synthetic compound that has a biological effect to inhibit or significantly reduce the expression of a gene. Consequently an “inhibitor of expression of a gene encoding a VLA-4 subunit” refers to a natural or synthetic compound that has a biological effect to inhibit or significantly reduce the expression of the gene encoding a VLA-4 subunit such as CD49d (alpha4 subunit) or CD29 (beta-1 subunit), preferably CD49d. According to the invention, such inhibitor can be called “inhibitor of VLA4 gene expression”.

Inhibitors of expression for use in the present invention may be based on anti-sense oligonucleotide constructs. Anti-sense oligonucleotides, including anti-sense RNA molecules and anti-sense DNA molecules, would act to directly block the translation of CD49d or CD29 mRNA by binding thereto and thus preventing protein translation or increasing mRNA degradation, thus decreasing the level of the given VLA-4 subunit, and thus activity, in a cell. For example, antisense oligonucleotides of at least about 15 bases and complementary to unique regions of the mRNA transcript sequence encoding a VLA-4 subunit can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of expression for use in the present invention. Gene expression can be reduced by contacting a subject or cell with a small double stranded RNA (dsRNA), or a vector or construct causing the production of a small double stranded RNA, such that the gene expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Ribozymes can also function as inhibitors of expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors of expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and preferably cells expressing VLA-4. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, 1990 and in Murry, 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

Example of antisense that may be used according to the invention are described in US 2009/0029931 which is incorporated by reference in is entirely. Other example also includes ATL1102 that is a second generation antisense inhibitor of CD49d (Myers et al; Antisense oligonucleotide blockade of alpha 4 integrin prevents and reverses clinical symptoms in murine experimental autoimmune encephalomyelitis, Journal of Neuroimmunology (2005) 160, 12-24).

Another object of the invention relates to a method for treating Duchenne Muscular Dystrophy comprising administering a subject in need thereof with a VLA-4 antagonist or an inhibitor of expression such as described above.

As used herein, the term “subject” denotes a mammal, such as a rodent, a feline, a canine, and a primate. Preferably, a subject according to the invention is a human.

VLA-4 antagonists or inhibitors of VLA4 gene expression may be administered in the form of a pharmaceutical composition, as defined below.

Preferably, said antagonist or inhibitor is administered in a therapeutically effective amount.

By a “therapeutically effective amount” is meant a sufficient amount of the VLA-4 antagonist or inhibitor of VLA4 gene expression to treat and/or to prevent Duchenne Muscular Dystrophy at a reasonable benefit/risk ratio applicable to any medical treatment.

It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

### Pharmaceutical Compositions

The VLA-4 antagonist or inhibitor of VLA4 gene expression may be combined with pharmaceutically acceptable excipients, and optionally sustained-release matrices, such as biodegradable polymers, to form therapeutic compositions.

The term “pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of viruses and microorganisms, such as mycoplasma, bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The VLA-4 antagonist or inhibitor of VLA4 gene expression of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with various of the other ingredients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will possibly occur depending on the condition of the subject being treated. The person responsible for administration will, in any event, determine the appropriate dose for the individual subject.

The VLA-4 antagonist or inhibitor of VLA4 gene expression of the invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

### Diagnostics Methods According to the Invention

A further aspect of the invention relates to a method for determining the prognosis of a subject affected with Duchenne Muscular Dystrophy wherein said method comprising a step consisting of determining the level of VLA-4^{high} T lymphocytes in a blood sample obtained from said subject.

According to the invention, the term “level” corresponds to the term “relative numbers” (particularly used in the example).

As used herein, the term “VLA-4^{high} T lymphocyte” refers to a T lymphocyte having a high expression of VLA-4 at its surface. According to the invention “high expression of VLA-4” means that said T lymphocyte expresses higher amounts of VLA-4 at their surface than a T lymphocyte obtained from a control group consisting of healthy individuals who are not affected with Duchenne Muscular Dystrophy. Typically said population of cells can be clearly indentified when methods of flow cytometry are performed. For instance, two populations may be distinguished in group of subjects. According to the invention, the T lymphocyte may be CD4 positive or CD8 positive.

Determining the amount of VLA-4^{high} T lymphocytes may be performed with any method well known in the art. For example the methods may consist in collecting a blood sample and using differential binding partners directed against VLA-4 and the specific surface markers of said T lymphocytes such as CD4 and CD8, wherein VLA-4^{high} T lymphocytes are bound by said binding partners to said surface markers. In a particular embodiment, the methods of the invention comprise contacting the blood sample with a set of binding partners capable of selectively interacting with VLA-4^{high} T lymphocytes present in the blood sample.

The binding partner may be an antibody that may be polyclonal or monoclonal, preferably monoclonal, directed against the specific surface markers of VLA-4^{high} T lymphocytes. In another embodiment, the binding partners may be a set of aptamers. Antibodies and aptamers may be raised by the methods as described above.

The binding partners of the invention such as antibodies or aptamers, may be labelled with a detectable molecule or substance, such as a fluorescent molecule, a radioactive molecule or any others labels known in the art. Labels are known in the art that generally provide (either directly or indirectly) a signal that can be quantified.

As used herein, the term “labelled”, with regard to the antibody or aptamer, is intended to encompass direct labelling of the antibody or aptamer by coupling (i.e., physically linking) a detectable substance, such as a radioactive agent or a fluorophore (e.g. fluorescein isothiocyanate (FITC) or phycoerythrin (PE) or Indocyanine (Cy5)) to the antibody or aptamer, as well as indirect labelling of the probe or antibody by reactivity with a detectable substance. An antibody or aptamer of the invention may be labelled with a radioactive molecule by any method known in the art. For example radioactive molecules include but are not limited radioactive atom for scintigraphic studies such as I123, I124, In111, Re186, Re188. Preferably, the antibodies against the surface markers are already conjugated to a fluorophore (e.g. FITC-conjugated and/or PE-conjugated). Examples include monoclonal anti-human CD62E-FITC, CDC105-FITC, CD51-FITC, CD106-PE, CD31-PE, and CD54-PE, available through Ancell Co. (Bayport, Minn.).

The aforementioned assays may involve the binding of the binding partners (ie. antibodies or aptamers) to a solid support. Solid supports which can be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like. The solid surfaces are preferably beads. Since VLA-4^{high} T lymphocytes have a diameter of roughly 3-8 μm, the beads for use in the present invention should have a diameter larger than 8 μm. Beads may be made of different materials, including but not limited to glass, plastic, polystyrene, and acrylic. In addition, the beads are preferably fluorescently labelled. In a preferred embodiment, fluorescent beads are those contained in TruCount™ tubes, available from Becton Dickinson Biosciences, (San Jose, Calif.).

According to the invention, methods of flow cytometry are preferred methods for determining the level of VLA-4^{high} T lymphocytes in the blood sample obtained from the subject. Said methods are well known in the art (See e.g., (1976) Herzenber et al. (1976) Sci. Amer., 234:108) For example, fluorescence activated cell sorting (FACS) may be therefore used to separate in the blood sample the desired microparticles. In another embodiment, magnetic beads (MACS) may be used to isolate VLA-4^{high} T lymphocytes. For instance, beads labelled with specific monoclonal antibodies may be used for the positive selection of VLA-4^{high} T lymphocytes. Other methods can include the isolation of VLA-4^{high} T lymphocytes by depletion of non VLA-4^{high} T lymphocytes (negative selection). For example, VLA-4^{high} T lymphocytes may be excited with 488 nm light and logarithmic green and red fluorescences of FITC and PE may be measured through 530/30 nm and 585/42 nm bandpass filters, respectively. The absolute number of VLA-4^{high} T lymphocytes may then be calculated through specific softwares useful in practicing the methods of the present invention. Typically, a fluorescence activated cell sorting (FACS) method such as described in Example 1 here below may be used to determining the levels of VLA-4^{high} T lymphocytes in the blood sample obtained from the subject.

Accordingly, in a specific embodiment, the method of the invention comprises the steps of obtaining a blood sample as above described; adding both labelled antibodies against surface markers that are specific to VLA-4, putting said prepared sample into a container having a known number of solid surfaces wherein the solid surfaces are labelled with a fluorescent dye; performing a flow cytometry analysis on the prepared sample in order to calculate the absolute and relative numbers of VLA-4^{high} T lymphocytes therein.

In one embodiment, the method of the invention may further comprise a step of comparing the level (or membrane density) of VLA-4 in VLA-4^{high} T lymphocytes with a predetermined value. As used herein, the term “predetermined value” refers to the levels (density) of VLA-4 in VLA-4^{high} T lymphocytes in the blood sample obtained from a selected population of subjects. For example, the predetermined value may be of the level of VLA-4 in VLA-4^{high} T lymphocytes obtained from subjects who lost their ambulation and became confined to a wheel chair before 10 years of age. The predetermined value can be a threshold value, or a range. For example, the predetermined value can be established based upon comparative measurements between subjects who lost their ambulation and became confined to a wheel chair before 10 years of age and subjects who lost their ambulation and became confided to a wheel chair after 10 years of age. A differential between the level of VLA-4 in VLA-4^{high} T lymphocytes determined by the method of the invention and the predetermined value is then indicative of the disease prognosis.

A further aspect of the invention relates to a method for determining the prognosis of a subject affected with Duchenne Muscular Dystrophy wherein said method comprises the step of analyzing a biological sample from said subject for:

(i) detecting the presence of a mutation in the gene encoding the CD49d (alpha 4 integrin chain) and/or CD29 (beta1 integrin chain) of VLA-4, and/or

(ii) analyzing the expression of the gene encoding the CD49d and/or CD29 of VLA-4.

Typical techniques for detecting a mutation in the gene encoding CD49d and/or CD29 of VLA-4 may include restriction fragment length polymorphism, hybridisation techniques, DNA sequencing, exonuclease resistance, microsequencing, solid phase extension using ddNTPs, extension in solution using ddNTPs, oligonucleotide assays, methods for detecting single nucleotide polymorphism such as dynamic allele-specific hybridisation, ligation chain reaction, mini-sequencing, DNA “chips”, allele-specific oligonucleotide hybridisation with single or dual-labelled probes merged with PCR or with molecular beacons, and others. Analyzing the expression of the gene encoding CD49d and/or CD29 of VLA-4 may be assessed by any of a wide variety of well-known methods for detecting expression of a transcribed nucleic acid or translated protein.

In a preferred embodiment, the expression of the gene encoding CD49d and/or CD29 of VLA-4 is assessed by analyzing the expression of mRNA transcript or mRNA precursors, such as nascent RNA, of said gene(s). Said analysis can be assessed by preparing mRNA/cDNA from cells in a blood sample from a subject, and hybridizing the mRNA/cDNA with a reference polynucleotide. The prepared mRNA/cDNA can be used in hybridization or amplification assays that include, but are not limited to, Southern or Northern analyses, polymerase chain reaction analyses, such as quantitative PCR (for example TaqMan), and probes arrays such as GeneChip™ DNA Arrays (for example AFFYMETRIX).

Advantageously, the analysis of the expression level of mRNA transcribed from the gene encoding CD49d and/or CD29 of VLA-4 involves the process of nucleic acid amplification, e.g., by RT-PCR (the experimental embodiment set forth in U.S. Pat. No. 4,683,202), ligase chain reaction (Barany, 1991), self sustained sequence replication (Guatelli et al., 1990), transcriptional amplification system (Kwoh et al., 1989), Q-Beta Replicase (Lizardi et al., 1988), rolling circle replication (U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

In another preferred embodiment, the expression of the gene encoding CD49d and/or CD29 of VLA-4 is assessed by analyzing the expression of the protein translated from said gene(s). Said analysis can be assessed using an antibody (e.g., a radio-labeled, chromophore-labeled, fluorophore-labeled, or enzyme-labeled antibody), an antibody derivative (e.g., an antibody conjugate with a substrate or with the protein or ligand of a protein of a protein/ligand pair (e.g., biotin-streptavidin)), or an antibody fragment (e.g., a single-chain antibody, an isolated antibody hypervariable domain, etc.) which binds specifically to the protein translated from the gene encoding CD49d (associated or not with CD29) and/or CD29 of VLA-4, preferably CD49d.

Said analysis can be assessed by a variety of techniques well known from one of skill in the art including, but not limited to, enzyme immunoassay (EIA), radioimmunoassay (RIA), Western blot analysis and enzyme linked immunoabsorbant assay (ELISA).

The method of the invention may comprise comparing the level of expression of the gene encoding CD49d and/or CD29 of VLA-4 in a blood sample from a subject with the normal expression level of said gene in a control. A significantly stronger level of expression of said gene in the blood sample of a subject as compared to the normal expression level is an indication that the subject has or is predisposed to developing a disease associated with an increased retinal vascular permeability. The “normal” level of expression of the gene encoding CD49d and/or CD29 of VLA-4 is the level of expression of said gene in a blood sample of a subject not afflicted by any disease associated with an increased retinal vascular permeability. Preferably, said normal level of expression is assessed in a control sample (e.g., sample from a healthy subject, which is not afflicted by any disease associated with an increased retinal vascular permeability) and preferably, the average expression level of said gene in several control samples.

According to the invention, the subjects having a poor prognosis may be then treated with a VLA-4 antagonist or inhibitor of VLA-4 gene expression as described above so as to slow down the progression of the disease.

A further aspect of the invention relates to use of VLA-4 as a biomarker of Duchenne muscular dystrophy prognosis.

### Example

Methods

Study Protocol and Patients.

Over a period of 2 years and 6 months we carried out both a descriptive and an observational study of 52 patients in whom the genetic diagnosis of DMD had been confirmed. The patients came from the Neurology and Cardiology Services at the Institute of Paediatrics of the Federal University of R10 de Janeiro, and which is a reference center for Duchenne muscular dystrophy in Brazil. The age of the patients ranged from 5 to 17 years. A control group of the same age and sex was obtained from the Pediatric Ambulatory service at the same Institute of Paediatrics, as well as from healthy volunteers.

Patients with any co-morbidity that could interfere with the immunologic status as well as those that refused to participate were excluded from our study. The co-morbidities were defined by analyzing the clinical history of the patients, by clinical examination and by laboratory exams when necessary. From the total of 52 DMD patients, 4 were excluded from the study.

All the procedures were approved by the ethical committee of the Institute of Pediatrics, (N° 196/96); and by the ethical committee of the Necker Hospital in Paris, France.

Description of Patient Groups.

The patients with DMD were sub-divided into three different groups according to their ability to walk an established distance of ten meters. The sub-groups were defined as: a) able to walk 10 meters in less than ten seconds (speed: >1 m/s); b) able to walk 10 meters in ten seconds or more (speed: ≦1 m/s) and c) unable to walk.

We also carried out a prospective study in which the group of DMD patients who were able to walk at a speed of ≦1 m/s were followed until they lost their ability to walk. If this was before 10 years of age then it was considered as a bad prognosis and if it was after 10 years of age it was considered as a rather good prognosis.

In a retrospective study, the group of patients who had lost deambulation was also subdivided into two different sub-groups according to the age at which the subject had stopped walking: before (bad prognosis) or after ten years of age (good prognosis).

By defining these different groups we could analyze all of our parameters according to the severity of the disease.

All of the patients were treated with prednisone (1 mg/kg/day during the first 10 days of each month) from the time at which the diagnosis was confirmed until the patient was no longer able to walk.

Muscle Biopsies.

The muscle biopsies used in this study, were obtained from a different cohort of DMD patients as part of the routine diagnostic procedure (at the time of diagnosis of the disease), with informed consent. In general, the choice of the muscle for the biopsy, usually the quadriceps, was based on the physical examination, and this muscle should be able to generate a force of at least ⅗ according to the MRC scale.

Muscle specimens were obtained by surgical biopsy procedure by Dr. Desguerre in the Neurology Service of the Necker Hospital in Paris.

Muscle specimens were prepared for histological analysis in the Pathology Service, and were deep frozen in cooled isopentane in liquid nitrogen. In this retrospective part of the study the biopsies were also sub-divided into 2 groups: DMD patients who subsequently lost the ability to walk before ten years of age; and DMD patients who subsequently lost their walking ability after the age of 10 years.

Immunofluorescence.

Immunohistochemical analyses of the cellular infiltrates and the expression of extracellular matrix components in muscle tissue were made by immunofluorescence microscopy on 5 μm frozen sections. The sections were acetone fixed for 10 minutes and incubated (30 min at room temperature) with a blocking solution containing 5% normal sheep serum (Dako) and 10% normal human serum (Sigma Aldrish) diluted in PBS/BSA 1%. The sections were then incubated with a given primary antibody for 1 h at room temperature, washed with phosphate-buffered saline (PBS) and then incubated with the appropriate secondary antibody for 30 minutes. The secondary antibodies were conjugated to different fluorochromes. Following PBS washing, the specimens were mounted in moviol containing DAPI and analyzed using a fluorescent microscope. (Olympus, U-RFL-T).

Negative controls, in which secondary antibodies were used alone, did not generate any significant labeling. Images obtained after the quadruple staining for the inflammatory infiltrate or for fibronectin were analyzed using Metamorph software (Molecular Devices).

For the analyses of the absolute numbers of CD8⁺ and CD4⁺ cells we counted these populations in all microscopic fields containing inflammatory infiltrates in the muscle. The total number of cells counted was divided by the number of microscopic fields, which gave us the mean number of cells/field in each muscle section. In order to obtain a quantitative estimate of the amount of fibronectin present in the different biopsies the number of pixels obtained for the specific fibronectin staining was divided by the area of the field, thus giving the number of pixels/μm². The same method was used to carry out the quantitative analyses of VLA-4, but in these sections we selected specific (CD4⁺/HLA-DR⁺ or CD8⁺/HLA-DR⁺) lymphocytes and checked first for the expression of the VLA-4 CD49d subunit.

We analyzed all of the fields with inflammatory infiltrates in the muscle sections from all of the patients. For the expression of fibronectin, we also analyzed 3-5 fields without inflammatory infiltrate in each patient.

Blood Sample.

The blood samples for our study and the samples for the routine follow-up of the patients were obtained at the same time. The total volume was 12 ml: 2 ml of which was used for the routine follow-up, and 10 ml for our experiments, which included the analysis of the phenotype of the mononuclear cells and the migration assay.

### Cytofluorometric Analyses.

To characterize the different populations of mononucleated cells present in the blood sample, we used a range of specific antibodies to identify the phenotype of the cells and the molecules involved in migration. The mononucleated cells obtained from the blood were first incubated, in a 96 well plate, with 5% fetal calf serum for 20 minutes at 4° C. Thereafter, the sample was incubated with the appropriate primary antibodies diluted in PBS/2% fetal calf serum for 1 hour at 4° C. After washing, cells were fixed and analyzed by flow cytometry in a FacsAria® flow cytometer (Becton Dickinson, San Jose, USA) equipped with CellQuest software. A cell gate excluding cell debris and non-viable cells was determined using forward versus side scatter parameters, being confirmed in some experiments with the use of propidium iodide staining and immediate analysis of unfixed cells. Analyses were done after recording 10,000 to 40,000 events for each sample. The samples were analysed using WinMdi's or Sumitht's sofwares specific for flow cytometry analyses.

Antibodies.

For immunohistochemical analyses we used specific antibodies to characterize the inflammatory infiltrate in the muscle biopsies: CD3, CD4, CD8 (Dako Co. Carpinteria, USA), CD49d (Abcam), HLA-DR (Invitrogen). To detect the extracellular matrix molecule fibronectin, we used a polyclonal rabbit antibody (Dako).

Secondary goat anti-mouse or goat anti-rabbit antibodies conjugated to different fluorochromes (Alexa Fluor 594, Alexa Fluor 488, steptavidin Cy5—in the case of a biotinylated secondary antibody) were used to reveal antibody binding.

For the flow-cytometry studies we used the following monoclonal antibodies conjugated to different fluorochromes: anti-CD3, CD4, CD8, CD49d, CD49e, CD49f (Pharmingen/Becton-Dickinson, San Diego, USA). Isotype matched unrelated antibodies were used as negative controls. These antibodies were also obtained from Pharmingen.

Migration Assay.

The migratory responses of the T lymphocytes were measured using the Transwell system. Briefly, 5-μm pore size Transwell plates (Costar; Corning) were coated with 10 μg/ml of fibronectin or BSA (as a negative control), for 1 h at 37° C. and then blocked with 10 μg/ml of BSA. Mononucleated cells (10⁶ cells/100 μA of RPMI/1% BSA) were added in the upper chambers. After 16 h of incubation at 37° C. in a 5% CO₂ humidified atmosphere, migration was defined by counting the cells that had migrated to the lower chambers. Cells were then labeled with appropriate Abs and analyzed by flow cytometry.

In another set of experiments prior to the migration assay, the mononuclear cells were treated with the antibody anti-VLA-4 (R&D System Lille, Europe), that recognizes the α4 chain of this molecule (CD49d). In this experiment, 10⁶ mononuclear cells were pretreated for 10 minutes at 37° C. with 10 μA of the VLA-4 Ab, at a concentration of 1 μg/ml. The cells were then resuspended in 90 μA of RPMI/1% BSA and the migration assay was performed as described previously.

Statistical Analyses.

Results were analyzed using the student's T test or One-way Anova. Turkey's test was made when more than 2 groups were being analyzed together. Differences between the groups were considered statistically significant when p≦0.05. The evaluation of the risk factor was made using EpiInfo sofware.

Results

General Features of DMD Patients.

Over a period of three and a half years 67 DMD patients have been enrolled in the study of the blood samples. Eighteen patients were able to walk more than 1 m/s (26.8%), 20 walked less than 1 m/s (29.8%) and 29 (43.2%) were wheel chair bound and had no mobility. The control group was made up of 12 age and gender matched individuals. The age range of the DMD patients was between 5 to 17 years (mean: 9.2 yr and median: 8 yr) and the control group from 4 to 19 years of age (mean: 10.4 yr and median: 9 yr), with no significant differences between them (p=0.54).

For the muscle biopsies, we analyzed 4 patients who had lost their ability to walk before 10 years of age and 5 patients who had lost the ability to walk after 10 years of age. The biopsies were carried out between 3-7 years of age, and there was no significant difference in the age between the groups (p=0.90). The mean time from biopsy to the loss of ambulation was 3.3 years (±1.14) and 5.9 years (±0.62), in the groups that lost the ability to walk before or after 10 years of age, respectively. There was no significant difference between the groups (p=0.17).

Increase of VLA-4 Membrane Expression on T Cell Subsets from DMD Patients: Correlation with Disease Progression.

In a first set of experiments, we evaluated the membrane expression of CD49d (the α integrin chain of the fibronectin receptor VLA-4, comparing DMD patients with healthy individuals. We found a significant increase in the relative numbers of both CD4⁺VLA-4^{high} and CD8⁺/VLA-4^{high} T lymphocytes in the blood from DMD subjects. Interestingly, we did not see a higher expression of other members of the β1 integrin sub-family, namely VLA-5 (also a fibronectin receptor) and VLA-6 (a laminin receptor). These data are summarized in Table 1.

We further analyzed VLA-4 membrane expression according to different DMD sub-groups, comparing them among each other and with healthy controls (see Table 2). No significant differences between the groups were observed in terms of the relative numbers of VLA-4-positive cells (including low and high expressors). However, the relative numbers of CD4⁺VLA-4^{Hi} and CD8⁺VLA-4^{Hi} T cells were significantly higher in the DMD patients who took 10 seconds or more to walk 10 meters (n=20) or who were unable to walk (n=29), when compared with the control healthy group (n=12).

There was no difference between the patients who took less than 10 seconds to walk 10 meters (n=18) versus controls.

TABLE 1
Increase in the relative numbers of circulating
CD4⁺ and CD8⁺ T cell subsets expressing high
densities of VLA-4 in patients with Duchenne muscular dystrophy

| | Relative cell number (mean ± SE)^{a} | | |
|---|---|---|---|
| T cell subpopulation | Healthy | DMD | p value |
| CD4⁺/VLA-4^{Hi} | 22.85 ± 1.45 | 28.51 ± 0.99 | 0.01 |
| CD8⁺/VLA-4^{Hi} | 25.48 ± 1.55 | 34.39 ± 1.52 | 0.005 |
| CD4⁺/VLA-5^{Hi} | 34.95 ± 2.68 | 34.97 ± 1.46 | 0.87 |
| CD8⁺/VLA-5^{Hi} | 31.10 ± 2.26 | 34.04 ± 2.09 | 0.46 |
| CD4⁺/VLA-6^{Hi} | 30.53 ± 2.40 | 27.85 ± 1.41 | 0.36 |
| CD8⁺/VLA-6^{Hi} | 18.73 ± 1.79 | 18.34 ± 1.25 | 0.65 |

^{a}Data are presented as relative cell numbers of T cell subsets expressing high levels of a given VLA molecule.

TABLE 2
Increase in the relative numbers of circulating CD4⁺ and
CD8⁺ T cell subsets expressing high densities of VLA-4 in correlates
with disease progression in patients with Duchenne muscular dystrophy^{a}

| | Healthy (n = 12) | >1 m/s (n = 20) | ≦1 m/s (n = 18) | Unable to walk (n = 29) |
|---|---|---|---|---|
| TCD4⁺/VLA-4 | 90.77 ± 7.39 | 87.00 ± 9.92 | 91.37 ± 7.47 | 86.50 ± 9.50 |
| CD4⁺/VLA-4^{Hi} | 20.35 ± 0.84 | 20.92 ± 1.20 | 28.68 ± 0.94** | 34.86 ± 1.60** |
| TCD8⁺/VLA-4 | 98.32 ± 1.28 | 97.07 ± 3.54 | 97.67 ± 2.93 | 96.48 ± 3.14 |
| CD8⁺/VLA-4^{Hi} | 23.49 ± 2.45 | 24.44 ± 2.23 | 37.62 ± 2.48* | 39.67 ± 2.33** |
| TCD4⁺/VLA-5 | 87.40 ± 2.63 | 81.74 ± 5.65 | 84.82 ± 1.92 | 79.87 ± 3.37 |
| CD4⁺/VLA-5^{Hi} | 30.47 ± 2.02 | 22.78 ± 1.19* | 33.91 ± 3.09 | 36.99 ± 1.72 |
| TCD8⁺/VLA-5 | 86.99 ± 2.04 | 81.01 ± 6.74 | 83.27 ± 3.18 | 77.12 ± 4.19 |
| CD8⁺/VLA-5^{Hi} | 28.09 ± 2.92 | 27.40 ± 3.09 | 34.19 ± 4.66 | 39.74 ± 3.09* |
| TCD4⁺/VLA-6 | 81.48 ± 5.35 | 71.83 ± 5.29 | 82.49 ± 2.94 | 73.72 ± 3.53 |
| CD4⁺/VLA-6^{Hi} | 27.86 ± 1.45 | 21.35 ± 1.17* | 32.74 ± 3.27 | 30.52 ± 2.06 |
| TCD8⁺/VLA-6 | 79.59 ± 4.17 | 67.35 ± 7.80 | 74.74 ± 3.50 | 62.05 ± 5.03 |
| CD8⁺/VLA-6^{Hi} | 17.06 ± 1.13 | 15.77 ± 1.18 | 22.70 ± 3.38* | 18.21 ± 2.11 |

^{a}Data are presented as relative cell numbers of T cell subsets expressing high levels of a given VLA molecule.

We then subdivided the group of patients who were no longer able to walk and checked the age at which these children had become wheel chair bound, we observed higher percentages of CD4⁺/HLA-4^{Hi} and CD8⁺/HLA-4^{Hi} T cells in the group of patients who had lost ambulation before 10 years of age (n=15), when compared with the group who had become wheel chair bound at 10 years of age or later (n=14), with p values=0.0003 and 0.0001, respectively. Therefore, once again the relative numbers of CD4⁺/HLA-4^{Hi} and CD8⁺/HLA-4^{Hi} T cells correlated with the disease progression.

Since the group of patients being wheel chair bound was older than the other groups, it was necessary to examine the effect of age on the number of CD4⁺/VLA-4^{Hi} and CD8⁺/VLA-4^{Hi} T cells. This is relevant since it has been shown that the number of T cells varies according to age (Moraes-Pinto et al., 2005), although nothing has been described concerning VLA-4 expression. To exclude this bias we analyzed the percentage of these cells according to the age of the patients: no correlation was observed.

Considering that the expression of the VLA-4 on the surface of the T cells varied with disease progression, we hypothesized that high numbers of VLA-4^{Hi} T cells might be predictive for disease progression (i.e, the age at which the patients loose ambulation and become confined to a wheel chair). We then searched for a correlation between the CD4⁺/VLA-4^{Hi} and CD8⁺/VLA-4^{Hi} T cell counts obtained at the beginning of the study with the course of disease, as seen in the specific cohort that was followed prospectively. We showed significantly higher relative numbers of CD4⁺/VLA-4^{Hi} and CD8⁺/VLA-4^{Hi} T cells at the beginning of the disease in the group of patients that had lost ambulation before 10 years of age (n=11), as compared to the group where ambulation was lost at/after 10 years of age (n=11).

These results demonstrate that the relative numbers of VLA-4^{Hi} T cell subsets in the blood are not only related to the severity of the disease but can also predict the rate of disease progression. According to sensibility/specificity analyses, a patient with more than 33.25% of CD8⁺/VLA-4^{Hi} T cells has a 81% risk of becoming wheel chair bound before 10 years age, whereas those with 28.23% CD4⁺/HLA-4^{Hi} T cells has a 72% risk of losing ambulation before the age of 10 years.

VLA-4-Mediated T Cell Migratory Responses Correlate with Disease Progression.

Since higher numbers of CD4⁺/VLA4^{Hi} and CD8⁺/VLA-4^{Hi} T lymphocytes at an early age seems to be predictive of a faster disease progression, as seen by early loss of ambulation, and considering that the interaction between the VLA-4 and VCAM-1 on the surface of the endothelial cells is important for the transmigration of the T cells from the blood to an inflammatory tissue (Friedl and Weigelin, 2008), we next investigated, in a selected group of 10 individuals if there was a difference in the transendothelial migration of T cells from the DMD patients groups. Both CD4⁺/VLA4^{Hi} and CD8⁺/VLA-4^{Hi} T cells from the most severe DMD patients, who were wheel chair bound (n=4), tended to migrate faster than the those isolated either from the less severe DMD patients who were able to walk at a speed of >1 m/s (n=3) or from the healthy control group (n=3). However, with this relatively small groups of subjects, differences between severely affected versus less affected or healthy controls remained non-significant.

Another VLA-4 natural ligand, fibronectin, is known to be involved in intra-tissue leukocyte migration, including in inflammation (Korpos and Wu, 2009). We thus carried out cell migration assays in order to determine whether T lymphocytes from DMD patients would also migrate faster through a fibronectin lattice formed on the porous inserts of transwell migration chambers. Again, higher fibronectin-driven migratory responses, of both CD4⁺/VLA4^{Hi} and CD8⁺/VLA-4^{Hi} T cells, were correlated with both disease progression.

Considering that integrins are involved in leukocyte proliferation and survival (Abram and Lowell, 2009), and that the interaction of VLA-4 with fibronectin could induce lymphocyte proliferation, we checked if, after the 16 hours of the migration assay, we could observe any difference in the proliferation or death of the cells in the different groups of patients. However, no difference was observed for either of these parameters, further demonstrating that the enhancement of migratory transendothelial cell and fibronectin-driven responses actually represent differential migration patterns, highlighting that the more severe is the condition of the DMD patient, the faster is the corresponding T cell migration, through the vascular endothelium as well as within the muscular tissue.

Presence of VLA-4⁺ Activated T Lymphocytes in the DMD Muscular Inflammatory Infiltrate: Correlation with Disease Progression.

Since the relative numbers of circulating CD4⁺/VLA4^{Hi} and CD8⁺/VLA-4^{Hi} T lymphocytes were highest in DMD patients with the worst prognosis, and since both CD4⁺/VLA4^{Hi} and TCD8⁺/VLA-4^{Hi} T cells from these patients also exhibited an increased transendothelial and fibronectin-driven migratory responses, it was conceivable that increased numbers of cells bearing this phenotype would be found within the skeletal muscle. Firstly, we noticed higher numbers of CD8⁺ T cells in muscle biopsies of patients that had lost ambulation before 10 years of age (p=0.04). The numbers of CD4⁺ T cells in the intramuscular inflammatory, infiltrates, although clearly higher than the numbers of CD8⁺ T lymphocytes, did not differ from patients that had stopped to walk before or after the age of ten years.

When we analyzed the activation state and the expression of VLA-4 on the CD4⁺ and T CD8 cells in the inflammatory infiltrate, we found a significantly higher frequency of CD8/⁺VLA-4⁺HLA-DR⁺ cells in the muscle biopsies of the patients who had a rapid disease progression, with p=0.03. Since HLA-DR is a marker of activated T cells in humans, our data indicate that there are more activated CD8⁺ T cells bearing high densities of VLA-4 in the patients with a rapid progression when compared to the less severe group of DMD patients. Of interest, we also found in the lesions, increased amounts of fibronectin, implying that, in addition to the higher VLA-4 membrane expression on infiltrating T lymphocytes, the muscle tissue itself augments the deposition of the ligand, a pattern that was typical in those patients who lost ambulation before ten years of age.

When we compared the two different groups of patients divided according to the prognosis of the disease we observed a more heterogeneous deposition of fibronectin, with a higher deposition of this molecule co-localized within the area of inflammatory infiltrate in the group of patients who became wheel chair bounded before 10 years of age.

Anti-VLA-4 Monoclonal Antibody Blocks Transendothelial and Fibronectin-Driven Migration of T cells from DMD Patients.

We have shown above that an increased number of VLA4^{Hi} T cells is correlated with both disease severity and disease progression. Also, the higher VLA-4 expression at the surface of the T cells likely correlates with increased migration capacity compared to T cells isolated from healthy subjects or DMD subjects with a less severe disease progression. All these data suggested that VLA-4 could play an important role in both the inflammatory response as well as in the disease progression in DMD patients. Because the interaction between the VLA-4 and VCAM-1 in the surface of the endothelial cells is important for the transmigration of the T cells from the blood to the tissue (Friedl and Weigelin, 2008), and considering that once within the tissue the interaction of this integrin with fibronectin is important for the intratissular migration of these cells, we investigated whether we could modify the migration of T cells ex-vivo, by selectively blocking VLA-4. For this, we treated T cells obtained from different DMD groups with an antiCD49d (alpha-4 integrin chain) antibody prior to migration through fibronectin or through endothelial cells. We observed that this pre-treatment partially (and in some cases completely) blocked both transendothelial and fibronectin-driven migration of T cells from DMD patients. The effect was even more striking when we examined the migratory behavior of CD4⁺/VLA4^{Hi} and CD8⁺/VLA-4^{Hi} T lymphocyte subsets, independently of the severity of the disease (Tables 3-4).

TABLE 3
Differential decrease in transendothelial T cell migratory response
after treatment with anti-VLA-4 monoclonal antibody^{a}

Groups of DMD patients

| T cell subsets | ≦1 m/s | Unable to walk |
|---|---|---|
| CD4⁺ | 88.86 ± 11.70 | 72.61 ± 8.97 |
| CD4⁺/VLA4⁺ | 87.41 ± 12.92 | 71.72 ± 8.61 |
| CD4⁺/VLA4^{Hi} | 10.95 ± 5.47 | 19.40 ± 12.54 |
| CD8⁺ | 91.15 ± 19.87 | 76.35 ± 10.62 |
| CD8⁺/VLA4⁺ | 88.83 ± 18.63 | 73.16 ± 9.65 |
| CD8⁺/VLA4^{HI} | 9.62 ± 7.34 | 22.03 ± 9.18 |

^{a}The mononucleated cells obtained from the blood of DMD patients were pre-treated in vitro with the antibody anti-VLA-4 as described. Then, the migration of different subpopulations of T cells through endothelial cells was analyzed as described. The numbers showed in the Table represents the % of migration (±S.E.), calculated out of the migration values obtained after treatment with the Ig, which were considered as 100%.

TABLE 4
Differential decrease in fibronectin-driven T cell migratory
response after treatment with anti-VLA-4 monoclonal antibody^{a}

Groups of DMD patients

| T cell subsets | ≦1 m/s | Unable to walk |
|---|---|---|
| TCD4⁺ | 18.03 ± 18.03 | 71.17 ± 23.9 |
| TCD4⁺/VLA4⁺ | 18.78 ± 18.78 | 72.3 ± 26.51 |
| TCD4⁺/VLA4^{Hi} | No migration | 21.05 ± 17.58 |
| TCD8⁺ | 29.73 ± 21.07 | 62.60 ± 24.87 |
| TCD8⁺/VLA4⁺ | 19.43 ± 14.58 | 61.15 ± 23.93 |
| TCD8⁺/VLA4^{HI} | No migration | 6.52 ± 4.21 |

^{a}The mononucleated cells obtained from the blood of DMD patients were pre-treated in vitro with the antibody anti-VLA-4 as described. Then, the fibronectin-driven migration of different T cell subpopulations was analyzed as described. The numbers showed in the Table represent the % of migration (±S.E.), calculated out of the migration values obtained after treatment with the Ig, which were considered as 100%.

Conclusions

Considering both the importance of the interaction between the integrins and their ligands for the migration of T lymphocytes during inflammation, as well as the participation of the immune system in the pathogenesis of DMD, we decided to investigate the expression of integrins on the surface of T cells in patients with DMD at different stages of the disease.

Subdividing the patients into groups according to the severity of disease we demonstrate that an increase in the percentages of CD4⁺/VLA-4^{high} T lymphocytes and CD8⁺/VLA-4^{high} T lymphocytes is correlated to the severity of the disease in DMD patients. This was further corroborated by a significant increase of CD4⁺/VLA-4^{high} T lymphocytes and CD8⁺/VLA-4^{high} T lymphocytes in the DMD cohort followed prospectively who lost deambulation early.

Together these results suggest that this higher expression of VLA-4 on the surface of the T cells could be used as a biomarker for the prognosis of DMD.

In addition, the ex-vivo migration assay carried out with cells obtained, either from patients in the more severe stages of the disease or from those with the worst prognosis, showed that this VLA4^{high} T cell subpopulations showed a greater fibronectin-driven migratory response than the mild and the control groups. We also demonstrated that, the pre-treatment of the mononucleated cells, in vitro, with an antibody against VLA4 was able to partially or completely block the ability of these cells to migrate.

Consequently, we propose that in the more severely affected patients a higher number of cells migrate into the muscle due to this interaction of the VLA-4 integrin with the fibronectin. Once in the muscle, these cells could contribute to perpetuate and aggravate the lesion by releasing cytokines, thus providing an environment which will favor fibrosis development.

Since there is no treatment for DMD, except the corticotherapy that can improve motor symptoms for only a short period of time, and since VLA-4 has already been used as a target in other inflammatory diseases with good results, we suggest that VLA-4 represents a novel biomarker to identify the patients with the worst prognosis, and maybe in whom a more aggressive therapeutic approach could be tried. In addition, blocking VLA-4 represent a new therapeutic target for DMD.

### REFERENCES

Throughout this application, various references describe the state of the art to which this invention pertains. The disclosures of these references are hereby incorporated by reference into the present disclosure.
Barany F. Genetic disease detection and DNA amplification using cloned thermostable ligase. Proc Natl Acad Sci USA. 1991 Jan. 1; 88(1):189-93.
Brummelkamp T R, Bernards R, Agami R: A system for stable expression of short interfering RNAs in mammalian cells. Science 296:550-553, 2002
Carlos T M, Schwartz B R, Kovach N L, Yee E, Rosa M, Osborn L, Chi-Rosso G, Newman B, Lobb R, et al. Vascular cell adhesion molecule-1 mediates lymphocyte adherence to cytokine-activated cultured human endothelial cells. Blood 1990 Dec. 1; 76(11):2420.
Clayberger C, Krensky A M, McIntyre B W, Koller T D, Parham P, Brodsky F, Linn D J, Evans E L. Identification and characterization of two novel lymphocyte function-associated antigens, L24 and L25. J. Immunol. 1987 Mar. 1; 138(5):1510-4.
Colas P, Cohen B, Jessen T, Grishina I, McCoy J, Brent R: Genetic selection of peptide aptamers that recognize and inhibit cyclin-dependent kinase 2. Nature 380:548-550, 1996
Cole et al. “Monoclonal Antibodies and Cancer Therapy”, Alan R. Liss, Inc., 1985, pp. 77-96.
Cote R J, Morrissey D M, Houghton A N, Beattie E J, Jr., Oettgen H F, Old L J: Generation of human monoclonal antibodies reactive with cellular antigens. Proc Natl Acad Sci USA 80:2026-2030, 1983
Dore-Duffy et al., Frontiers in Cerebral Vascular Biology: Transport and Its Regulation, pp. 243-248. Eds. Drewes & Betz, Plenum, N.Y. 1993.
Elbashir S M, Harborth J, Lendeckel W, Yalcin A, Weber K, Tuschl T: Duplexes of 21-nucleotide RNAs mediate RNA interference in cultured mammalian cells. Nature 411:494-498, 2001
Guatelli J C, Whitfield K M, Kwoh D Y, Barringer K J, Richman D D, Gingeras T R. Isothermal, in vitro amplification of nucleic acids by a multienzyme reaction modeled after retroviral replication. Proc Natl Acad Sci USA. 1990 March; 87(5):1874-8.
Herzenberg L A, Sweet R G, Herzenberg L A. Fluorescence-activated cell sorting. Sci Am. 1976 March; 234(3):108-17.
Hannon G J: RNA interference. Nature 418:244-251, 2002
Jayasena S D: Aptamers: an emerging class of molecules that rival antibodies in diagnostics. Clin Chem 45:1628-1650, 1999
Kohler G, Milstein C: Continuous cultures of fused cells secreting antibody of predefined specificity. Nature 256:495-497, 1975.
Kriegler M. Assembly of enhancers, promoters, and splice signals to control expression of transferred genes. Methods Enzymol. 1990; 185:512-27.
Kwoh D Y, Davis G R, Whitfield K M, Chappelle H L, DiMichele L J, Gingeras T R. Transcription-based amplification system and detection of amplified human immunodeficiency virus type 1 with a bead-based sandwich hybridization format. Proc Natl Acad Sci USA. 1989 February; 86(4):1173-7.
Lagrota-Candido J., Canella I., Savino W., and Quirico-Santos T. Expression of extracellular matrix ligands and receptors in the muscular tissue and draining lymph nodes of mdx dystrophic mice. 1999. Clin. Immunol. 93 (2): 143-51.
Lizardi et al., Biol. Technology, vol. 6, p: 1197, 1988.
McManus M T, Haines B B, Dillon C P, Whitehurst C E, van Parijs L, Chen J, Sharp P A: Small interfering RNA-mediated gene silencing in T lymphocytes. J Immunol 169:5754-5760, 2002
Murry, “Methods in Molecular Biology,” vol. 7, Humana Press, Inc., Cliffton, N.J., 1991.
Sambrook J, Fritsch E F, Maniatis T. Molecular cloning: a laboratory manual, 2nd edition. Cold Spring Harbor Laboratory Press, Cold Spring Harbor, N.Y. 1989.
Tuerk C, Gold L: Systematic evolution of ligands by exponential enrichment: RNA ligands to bacteriophage T4 DNA polymerase. Science 249:505-510, 1990
Tuschl T, Zamore P D, Lehmann R, Bartel D P, Sharp P A: Targeted mRNA degradation by double-stranded RNA in vitro. Genes Dev 13:3191-3197, 1999

1-10. (canceled)

11. A method of treating Duchenne Muscular Dystrophy in a patient in need thereof, comprising the step of
administering to said patient a VLA-4 antagonist.

12. The method of claim 11, wherein said VLA-4 antagonist is selected from the group consisting of a low molecular weight antagonist, a peptide, an antibody and an aptamer.

13. The method of claim 11, wherein said antibody is Natalizumab®.

14. A method of treating Duchenne Muscular Dystrophy in a patient in need thereof, comprising the step of
administering to said patient an inhibitor of expression of a gene encoding a VLA-4 subunit.

15. The method of claim 14, wherein said inhibitor of expression is an inhibitor of expression of the gene encoding CD49d (alpha4 integrin subunit).

16. The method of claim 14, wherein said inhibitor is selected from the group consisting of antisense RNA molecules, antisense DNA molecules, small inhibitory RNAs (siRNAs), short hairpin RNAs and ribozymes.

17. A method for determining the prognosis of a subject affected with Duchenne Muscular Dystrophy, comprising the steps of
i) obtaining a blood sample from said subject;
ii) determining a level of VLA-4^{high} T cells in the blood sample;
iii) comparing the level of VLA-4^{high} T lymphocytes in the blood sample with a predetermined value, and
iv) concluding that said subject has a poor prognosis when the level of VLA-4^{high} T cells in the blood sample is greater than the predetermined value.

18. The method according to claim 17, wherein said step of determining further comprises the steps of
adding to the blood sample labeled antibodies against surface markers that are specific to VLA-4^{high} T lymphocytes;
putting the blood sample into a container having a known number of solid surfaces wherein the solid surfaces are labeled with a fluorescent dye; and
performing fluorescence-activated cell sorting (FACS) flow cytometry on the blood sample in order to calculate the absolute number of VLA-4^{high} T cells therein.

19. A method for determining the prognosis of a subject affected with Duchenne Muscular Dystrophy comprising the steps of
obtaining a biological sample from the subject; and
analyzing the biological sample using one or both of the following steps:
i) detecting the presence of a mutation in one or both of a) a gene encoding CD49d (alpha4 integrin chain) of VLA-4 and b) a gene encoding CD29 (beta-1 integrinn chain) of VLA-4; and
ii) analyzing the expression of one or both of a) a gene encoding CD49d (alpha4 integrin chain) of VLA-4 and b) a gene encoding CD29 (beta-1 integrinn chain) of VLA-4.

20. A pharmaceutical composition for use in the treatment of Duchenne Muscular Dystrophy, said pharmaceutical composition comprising
a pharmaceutically acceptable carrier, and either
a VLA-4 antagonist, or
an inhibitor of expression of a gene encoding a VLA-4 subunit.

21. The pharmaceutical composition of claim 20, wherein said VLA-4 antagonist is selected from the group consisting of a low molecular weight antagonist, a peptide, and an aptamer.

22. The pharmaceutical composition of claim 20, wherein said inhibitor of expression of a gene encoding a VLA-4 subunit is an inhibitor of expression of a) a gene encoding CD49d (alpha4 integrin chain) of VLA-4 orb) a gene encoding CD29 (beta-1 integrinn chain) of VLA-4.

23. The method of claim 22, wherein said inhibitor is selected from the group consisting of antisense RNA molecules, antisense DNA molecules, small inhibitory RNAs (siRNAs), short hairpin RNAs and ribozymes.
